(19) United States
(12) Patent Application Publication
(10) Pub. No.: US 2012/0315231 A1
(43) Pub. Date: Dec. 13, 2012

(54) COMPOSITION OF NAIL POLISH REMOVER

(76) Inventor: Hsien-Wen LIN, Chiayi City (TW)

(21) Appl. No.: 13/156,733

(22) Filed: Jun. 9, 2011

(51) Int. Cl. A61K 8/49; A61K 8/92; A61Q 3/00; A61Q 3/04; A61Q 17/00; A61K 8/72; A61K 8/97

(52) U.S. Cl. 424/61; 510/118

(57) ABSTRACT

The present invention discloses a composition of a nail polish remover to overcome the drawbacks of conventional nail polish removers that contain acetone, have irritating smells, and cause corrosive damages. The composition of the present invention includes gamma-butyrolactone with a weight percentage of 10% to 95%, water with a weight percentage of 2.3899% to 52.9% and an additive with a weight percentage of 2.6101% to 37.1%. The main composition used for removing nail polish is γ-butyrolactone which has no irritating smell and less damage to nails.

### BACKGROUND OF THE INVENTION

(a) Field of the Invention

The present invention relates to a composition of a nail polish remover, in particular to a polish nail remover lotion or gel containing γ-butyrolactone.

(b) Description of the Related Art

Modern women pay increasingly more attention to their appearance and dress, and nail cosmetic gradually becomes one of the necessary items. In general, a nail polish includes an organic paint or organic pigment which cannot be removed by water easily, and it is necessary to use a nail polish remover containing a volatile organic compound (VOC) such as isopropanol, acetone, methanol and ethyl acetate provided for dissolving the organic paint and being carried away by water, and the nail polish remover containing acetone may corrode, weaken, and facture the nails easily or may even induce bacteria and or mildews while removing the nail polish. A long time of use may result in onychorrhexis (also know as brittle nails) that jeopardize our health and safety.

In general, the nail polish remover is in a liquid form, and the liquid nail polish remover with low viscosity and high liquidity cannot be applied directly onto the nail, and thus a user needs to wet a cotton pad with the nail polish remover and rub the nails back and forth by the wet cotton pad. However, such method may stain the user's skin easily, and the user's skin may absorb too much organic solvent. As a result, the content of the organic solvent may cause a risk of intoxication after a long time of use, and rubbing the nails back and forth may further corrode the nails.

In summation of the description above, the liquid nail polish remover with a primary ingredient of acetone which is not suitable for the nail polish remover.

### SUMMARY OF THE INVENTION

To overcome the aforementioned technical problem, the present invention provides a nail polish remover capable of removing a nail polish quickly and reducing the damage of the nail caused by the ingredients of the nail polish remover. The inventor of the present invention based on years of experience in the related industry to conduct extensive researches and experiments, and finally developed a composition of a nail polish remover comprising: gamma-butyrolactone of 10% to 95% by weight, water of 2.3899% to 52.9% by weight, and an addition of 2.6101% to 37.1% by weight.

The additive contains 2.5% to 25% by weight of an emulsifier to make the nail polish remover of the present invention in form of a lotion, and the emulsifier preferably contains 0.5% to 5% by weight of glyceryl stearate, 0.5% to 5% by weight of stearic acid, 0.5% to 5% by weight of Arlacel 165 (Glyceryl stearate & PEG-100 Stearate), 0.5% to 5% by weight of cetyl-stearyl alcohol, and 0.5% to 1% by weight of hydroxyethyl acrylate/sodium acryloyldimethyl taurate copolymer. The emulsifier can further contain 0.01% to 4% by weight of a bee wax.

The additive further contains 0.05% to 2% by weight of a thickener, such that the composition of the present invention is form of a gel, and the thickener is preferably a hydrophilic thickener such as hydroxypropyl guar.

The additive further contains a plant extract, an antibacterial agent, an anti-mildew agent, an essence, and a pigment. The present invention has the following effects:

1. In the composition of the present invention, γ-butyrolactone is used as the main ingredient of the nail polish remover, and γ-butyrolactone has no irritating smell, and causes less damage to the nails.

2. The nail polish remover of the present invention is made in form of a lotion or a gel to increase the viscosity, such that the nail polish remover will not wet other positions of the body of the user, and has the effects of causing less damage to the nails, lowering the absorption rate, creating less harm to the user's health.

3. The additive of the present invention can further include a plant extract such as a cucumissativus extract with an anti-oxidation and anti-ultraviolet effects to protect the nails, an antibacterial agent and an anti-mildew agent for cleaning the bacteria and mildews accumulated on the nail surface, or an essence and a pigment to enhance the visual and odor effects and improve the product competitiveness.

### BRIEF DESCRIPTION OF THE DRAWINGS

No figure is provided.

### DETAILED DESCRIPTION OF THE PREFERRED EMBODIMENTS

The technical characteristics and effects of the present invention will become apparent from the detailed description of the following preferred embodiments.

The composition of a nail polish remover of the present invention comprises: 10% to 95% by weight of gamma-butyrolactone, 2.3899% to 52.9% by weight of water, and 2.6101% to 37.1% by weight of an additive.

In this embodiment, the additive contains 2.5% to 25% by weight of an emulsifier, such that the nail polish remover of the present invention is in form of a lotion, and the emulsifier contains 0.5% to 5% by weight percentage of glyceryl stearate, 0.5% to 5% by weight of stearic acid, 0.5% to 5% by weight of Arlacel 165 (Glyceryl stearate & PEG-100 Stearate), 0.5% to 5% by weight of cetyl-stearyl alcohol and 0.5% to 1% by weight of a hydroxyethyl acrylate/sodium acryloyldimethyl taurate copolymer. In this embodiment, the emulsifier further contains 0.01% to 4% by weight of a bee wax. In Tables 1 and 2, two types of compositions (% by weight) of the present invention in the lotion form and the time required for removing the nail polish are listed.

TABLE 1

| Composition | Percentage (%) |
|---|---|
| gamma-butyrolactone | 90 |
| Glyceryl Stearate | 0.5 |
| Stearic acid | 0.5 |
| Arlacel 165 (Glyceryl stearate & PEG-100 Stearate) | 0.5 |
| Cetyl-Stearyl Alcohol | 0.5 |
| bee wax | 0.01 |
| Propylene Glycol | 2 |
| Vitamin E acetate | 0.01 |
| Aqua | 5.28 |
| Cucumissativus extract | 0.5 |
| Hydroxyethyl Acrylate/Sodium Acryloyldimethyl | 0.1 |
| Taurate Copolymer | |
| Fragrance | 0.1 |
| Chlorhexidine Gluconate | 0.005 |
| Piroctone olamine | 0.005 |
| total | 100 |

Time of the product remaining on the nail: 10 seconds

TABLE 2

| Composition | Percentage (%) |
|---|---|
| gamma-butyrolactone | 85 |
| Glyceryl Stearate | 1 |
| Stearic acid | 0.5 |
| Arlacel 165 (Glyceryl stearate & PEG-100 Stearate) | 0.5 |
| Cetyl-Stearyl Alcohol | 1 |
| bee wax | 0.01 |
| Propylene Glycol | 2 |
| Vitamin E acetate | 0.01 |
| Aqua | 8.765 |
| Cucumissativus extract | 1 |
| Hydroxyethyl Acrylate/Sodium Acryloyldimethyl | 0.1 |
| Taurate Copolymer | |
| Fragrance | 0.1 |
| Chlorhexidine Gluconate | 0.01 |
| Piroctone olamine | 0.01 |
| FD&C Red No. 6 | 0.005 |
| Total | 100 |

Time of the product remaining on the nail: 15 seconds

In the composition of the nail polish remover lotions of the aforementioned embodiments, the time required for removing the nail polish is approximately 10 to 280 seconds depending on different gamma-butyrolactone contents. In a modification of the present invention, the addition further contain 0.05% to 2% by weight of a thickener to make the nail polish remover of the present invention in form of a gel, and the thickener is preferably a hydrophilic thickener such as hydroxypropyl guar. In Tables 3 and 4, two types of compositions (% by weight) of the present invention in the gel form and the time required for removing the nail polish are listed.

TABLE 3

| Composition | Percentage (%) |
|---|---|
| gamma-butyrolactone | 90 |
| Glycerol | 0.1 |
| Polyethylene Glycol 400 | 0.1 |
| Vitamin E acetate | 0.01 |
| Aqua | 9.27975 |
| Cucumissativus extract | 0.1 |
| Hydroxypropyl guar | 0.05 |
| Chlorhexidine Gluconate | 0.01 |
| Piroctone olamine | 0.05 |
| FD&C Red No. 6 | 0.00025 |
| Fragrance | 0.3 |
| total | 100 |

Time of the product remaining on the nail: 10 seconds

TABLE 4

| Composition | Percentage (%) |
|---|---|
| gamma-butyrolactone | 80 |
| Glycerol | 0.7 |
| Polyethylene Glycol 400 | 0.3 |
| Vitamin E acetate | 0.1 |
| Aqua | 17.2695 |
| Cucumissativus extract | 1 |
| Hydroxypropyl guar | 0.2 |
| Chlorhexidine Gluconate | 0.03 |
| Piroctone olamine | 0.1 |
| FD&C Red No. 6 | 0.00025 |
| FD&C Yellow No. 4 | 0.00025 |
| Fragrance | 0.3 |
| total | 100 |

Time of the product remaining on the nail: 15 seconds

In the composition of the nail polish remover gels of the aforementioned embodiments, the time required for removing the nail polish is approximately 10 to 300 seconds depending on different gamma-butyrolactone contents.

In the aforementioned embodiments, γ-butyrolactone is used as a main ingredient, and γ-butyrolactone has no irritating smell and less damage to the nails, and the lotion or gel can increase the viscosity, such that a user can apply an appropriate quantity of lotion or gel to the nail surface, let the lotion or gel sit still on the nail surface for an appropriate time, and finally remove the nail polish by tissue paper to complete removing the nail polish without wetting other positions of the user's body or severely damaging the nails by back and forth rubbing. In addition, the invention has a lower absorption rate, and cause a less harm to the user's health. In each embodiment, the additive contains a plant extract, an antibacterial agent (chlorhexidine gluconate, CH), an anti-mildew agent (piroctone olamine, PO), an essence, pigment, wherein the plant extract can be a cucumissativus extract having the anti-oxidation and anti-ultraviolet effects for protecting the nails. The antibacterial agent and the anti-mildew agent of the additive are provided for cleaning the bacteria and mildews accumulated on the nail surface, and the essence and the pigment are provided for enhancing the visual and odor effects and improve the product competitiveness.

While the invention has been described by means of specific embodiments, numerous modifications and variations could be made thereto by those skilled in the art without departing from the scope and spirit of the invention set forth in the claims.

I claim:

1. A composition of a nail polish remover, comprising:
gamma-butyrolactone with a weight percentage of 10% to 95%;
water with a weight percentage of 2.3899% to 52.9%; and
an additive with a weight percentage of 2.6101% to 37.1%, wherein the weight percentage is based on the total weight of the nail polish remover.

2. The composition of a nail polish remover as recited in claim 1, wherein the additive is comprised of an emulsifier with a weight percentage of 2.5% to 25% for making the composition of the polish nail remove in form of a lotion.

3. The composition of a nail polish remover as recited in claim 2, wherein the emulsifier is comprised of glyceryl stearate with a weight percentage of 0.5% to 5%, stearic acid with a weight percentage of 0.5% to 5%, Arlacel 165 (glyceryl stearate & PEG-100 stearate) with a weight percentage of 0.5% to 5%, cetyl-stearyl alcohol with a weight percentage of 0.5% to 5%, and hydroxyethyl acrylate/sodium acryloyldimethyl taurate copolymer with a weight percentage of 0.5% to 1%.

4. The composition of a nail polish remover as recited in claim 3, wherein the emulsifier is further comprised of a bee wax with a weight percentage of 0.01% to 4%.

5. The composition of a nail polish remover as recited in claim 1, wherein the additive is comprised of a thickener with a weight percentage of 0.05% to 2% to make the composition of the nail polish remover in form of a gel.

6. The composition of a nail polish remover as recited in claim 5, wherein the thickener is hydroxypropyl guar.

7. The composition of a nail polish remover as recited in claim 1, wherein the additive is further comprised of a plant extract, an antibacterial agent, an anti-mildew agent, an essence and a pigment.

8. The composition of a nail polish remover as recited in claim 2, wherein the additive is further comprised of a plant extract, an antibacterial agent, an anti-mildew agent, an essence and a pigment.

9. The composition of a nail polish remover as recited in claim 3, wherein the additive is further comprised of a plant extract, an antibacterial agent, an anti-mildew agent, an essence and a pigment.

10. The composition of a nail polish remover as recited in claim 4, wherein the additive is further comprised of a plant extract, an antibacterial agent, an anti-mildew agent, an essence and a pigment.

11. The composition of a nail polish remover as recited in claim 5, wherein the additive is further comprised of a plant extract, an antibacterial agent, an anti-mildew agent, an essence and a pigment.

12. The composition of a nail polish remover as recited in claim 6, wherein the additive is further comprised of a plant extract, an antibacterial agent, an anti-mildew agent, an essence and a pigment.
